(19) United States
(12) Patent Application Publication
(10) Pub. No.: US 2026/0023045 A1
(43) Pub. Date: Jan. 22, 2026

(54) METHODS FOR CHARACTERIZING A POLYMER

(71) Applicant: GOODMAN CONSULTING GROUP, LLC, Tucson, AZ (US)

(72) Inventor: Thomas C. Goodman, Tucson, AZ (US)

(73) Assignee: GOODMAN CONSULTING GROUP, LLC, Tucson, AZ (US)

(21) Appl. No.: 19/252,966

(22) Filed: Jun. 27, 2025

Related U.S. Application Data
(60) Provisional application No. 63/673,384, filed on Jul. 19, 2024.

(51) Int. Cl. G01N 27/447; G01N 27/30; G01N 33/68

(52) U.S. Cl. CPC G01N 27/447; G01N 27/30; G01N 33/6818

(57) ABSTRACT

The present invention provides methods for characterizing the composition of polymers. Common to these methods, the subunits of a charged polymer strand are moved by the force of electrophoresis across closely spaced electrodes so as to produce tunneling currents modulated by their passage and indicative of their chemical structure. Comparisons of the electronic signals thus obtained to those of known standards that have undergone similar electrophoresis are made so as to determine the composition of the polymer and the linear sequence of its subunits.

The invention described here may be used for: the detection and identification of biological agents that may be pathogenic; the sequencing of small linear RNAs that may be present in cells and those that have been linearized by the methods described herein; and the sequencing of linear genomic DNA, mRNAs, linear non-coding RNAs, and polypeptides.

### CROSS-REFERENCE TO RELATED APPLICATION

This application claims priority to U.S. Provisional Application No. 63/673,384 that was filed on Jul. 19, 2024. The entire content of the applications referenced above is hereby incorporated by reference herein.

### STATEMENT REGARDING FEDERALLY SPONSORED RESEARCH

This invention was made with government support under Grant Award No. 2230484 awarded by National Science Foundation. The government has certain rights in the invention.

### BACKGROUND

Present methods for detecting biological agents depend on knowing ahead of time what agent is expected to be present. For example, the polymerase chain reaction (PCR) is exquisitely sensitive, but its primers need to be designed ahead of time to hybridize to specific sequences in the biological agent to be detected. Immunoassays, especially those employing monoclonal antibodies, can be highly specific, but again, ahead of time, those antibodies have to be raised against and selected to react with specific antigens that are characteristic of the biological agent to be identified.

In contrast to this situation, it would be desirable to have a method to detect biological agents without having to know ahead of time what specific nucleic acid sequences or antigenic determinants are present. This could be accomplished by direct, single-molecule nucleic acid sequencing of the species present in a sample to be analyzed. The realization of such a method presents numerous challenges, and progress toward overcoming these has been reviewed [1].

For direct, single-molecule sequencing, the nucleic acid polymer must be examined or measured at the level of its individual nucleotide bases. To date, this has best been accomplished by drawing the polymer through a biological nanopore that separates two ionic solution-filled chambers and measuring changes in the ionic current observed through the nanopore as the polynucleotide transits [2]. This is an elegant approach exploiting the natural structures and specificity available in protein nanopores, but it suffers from three deficiencies.

First, and most importantly, the current levels observed during polynucleotide transit may come about from the combined contributions of multiple nucleotide bases in the polymer strand. Indeed, it is possible that as many as five adjacent bases may simultaneously influence a given observed current level measurement [1]. Second, the protein nanopore, even when modified [3], provides only an imperfect ratchet mechanism to control the transit of nucleotide bases. When a series of nucleotide bases moves through the pore too quickly to be separately identified, even with multiple reads—and thus doing away with the notion of “single-molecule” sequencing—this can lead to uncertainties in base calling [4]. Third, and a general deficiency of all sequencing methods employing PCR or other strand copying mechanisms, is that information about post-transcriptional or post-replication base modifications, as well as about any minor or chemically modified bases that may be present in the nucleic acid polymer is lost. Such information may be biologically or even clinically important and improved technologies are needed to capture it [5].

Solid state nanopores would appear to be an attractive alternative to protein nanopores and might be capable of overcoming some of the latter's disadvantages. Unfortunately, a solid-state nanopore is typically only about 5 nm, or less, in diameter and easily closes or clogs. In addition, even when the pore is open, the electric field intensity at the pore is so high that polynucleotides are observed to transit at from 1 to 10 million bases per second. This far exceeds the ability of even the best electronics available to distinguish base-specific current fluctuations.

Finally then, while solid state nanopores have largely been abandoned for nucleic acid sequencing applications [6], still a reimagination of their geometry, combined with quantum mechanics-based electronic sensing, may enable the creation of a new structure and device to overcome the disadvantages and limitations described above. The present application shows how this may be accomplished.

### SUMMARY

The present invention eliminates the closed circular nanopore and replaces it in certain embodiments with any of: an open, flat, curved, or notch-like structure. This type of structural modification dramatically reduces the electric field experienced by the polynucleotide in transit and is less likely to become clogged or closed. In contrast to protein nanopores, the open, solid-state structure described below may accommodate amplification electronics on the sensor chip itself in very close connection to the sensor elements [7] and thus enable more accurate and faster measurements. In addition, rather than measuring the current blockage of a protein nanopore, which experimentally provides signal current changes of about 10 to 30 picoamps as a readout, the ability to place sensing electrodes within about 2 nm of each other in the solid-state structure allows for the observation of tunneling currents. Tunneling microscopy is observed with tunnel currents in the 1 to 10 nanoampere range, and the exponential dependence of tunneling on distances and barrier heights [8] can be expected to increase this range and improve the contrast between different bases passing across the tunneling electrodes.

Certain embodiments provide a method of characterizing the composition of a charged polymer comprising:
introducing the polymer into an electrically conductive medium,
applying a voltage to said conductive medium that causes said polymer to move across closely spaced measurement electrodes so that a tunneling current between said closely spaced measurement electrodes is produced,
measuring time-dependent changes in said tunneling current, and
correlating said changes in said tunneling current to the composition of said polymer.

In certain embodiments, the polymer is a polynucleotide.

In certain embodiments, the polynucleotide is substantially single-stranded.

In certain embodiments, the polymer is a polypeptide.

In certain embodiments, the closely spaced measurement electrodes have been processed by the electro-polishing or electro-deposition of a conducting material on their surfaces.

In certain embodiments, the first derivative of the time-dependent changes in the tunneling current (dI/dt) is calculated to determine the composition of the polymer.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Tunneling electrode pair at the working face of the comb root (not to scale).

FIG. 2 diagrams how a base (guanine ribonucleotide—G) passes across the conducing electrodes (FIG. 2, top panel) and presents a side-view potential energy diagram (FIG. 2, bottom panel) of how the tunneling current may be thought of during such an event.

FIGS. 3A-3E. FIG. 3A shows the 1×4 cm silicon chip. FIG. 3B shows the placement of the first electrode; FIG. 3C shows the addition of the insulating layer; FIG. 3D show the second electrode; and FIG. 3E shows the finished chip inserted into the aqueous electrophoresis buffer (green wavy line and shading). Rectangles at bottom of the Figure depict the edge view of the chip. The red arrow indicates the direction of nucleic acid movement across the tunneling electrodes. The Figures are not to scale.

FIG. 4. Chip produced by the fabrication process are scored so as to be able to be sawed or broken off easily.

FIG. 5 shows the detector chip assembly clamped or glued to a support beam that allows them to be suspended in an electrophoresis solution (light blue shading) in the buffer chamber. Electrophoresis electrodes (not shown) are placed just below the buffer line in front and behind the detector chip assembly.

FIG. 6. Analogous to the rightmost panel of FIG. 1 above, and not to scale, the pairs of tunneling current sensing electrodes are each represented by a single light blue line; and the “hold and release” electrodes are shown as the longer, thicker green lines. Both of these are drawn at the top face (also, the “working face”) of the channel root, reflecting their introduction by vapor deposition/nanolithography on one surface of the original silicon substrate. Both faces can be used and multiple chips stacked, limited by the size, mass, and heat generation of the associated amplification and A/D conversion electronics.

FIGS. 7A-7C. FIG. 7A. Transit of the nucleic acid (red) avoids the tunneling current electrodes (light blue). The black line represents the top edge of the tunneling chip. FIG. 7B. Proportional voltage (˜50/50 drawn here, but varying from chip to chip and determined empirically) allows the nucleic acid to transit the tunneling electrodes. FIG. 7C. Voltage skewed to the left+electrode causes the nucleic acid's path (in red) to move at ˜45° to the tunneling electrodes.

FIG. 8. Cross-section of tunneling chip showing the variable angle at which a nucleic acid polymer, for example, can be drawn across the sensor chip.

FIG. 9. Idealized data output from the operation of the invention with a polynucleotide.

### DETAILED DESCRIPTION

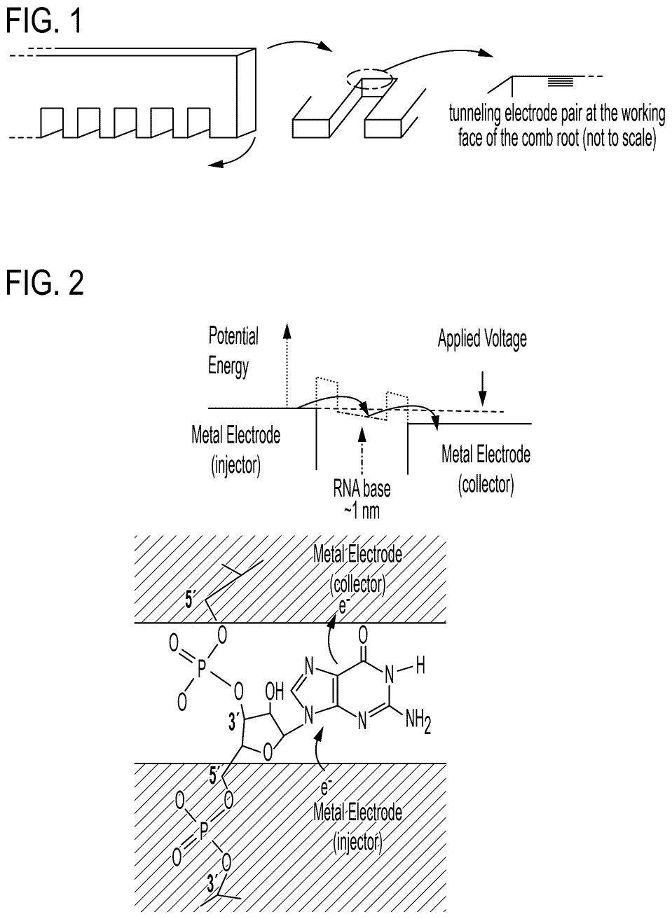

The invention begins with the creation of a notch- or comb-like channel through which a nucleic acid strand or other charged polymer can be moved by electrophoresis. An embodiment of this construct with multiple rectangular notches is shown in FIG. 1. Other embodiments, depending on application constraints, may contain a V-shaped notch or possibly a smooth concave surface, each with essentially the same embedding of tunneling current detection electrodes as shown.

During its movement through this channel, the base subunits of a nucleic acid strand, for example, will be dragged across a closely spaced pair of electrodes (or multiple pairs of electrodes, as described herein) embedded at the root surface of a notch. As an individual base transits a pair of electrodes, a tunneling current is produced and measured by the electronics of the system.

Tunneling is a well-known quantum mechanical effect. It allows for particles to enter into regions of space that would normally be forbidden by classical Newtonian mechanics. In the tunneling phenomenon, the quantum mechanical spatial waveform of a particle encounters a barrier and undergoes exponential decay. For an electron, it is envisioned that if the barrier is such that the waveform does not decay to zero during its propagation through the barrier, the electron appears on the other side of the barrier and is said to have “tunneled” through it. The movement of electrons experimentally observed in this phenomenon is the tunneling current [9].

FIG. 2 diagrams how a base (guanine ribonucleotide—G) passes across the conducing electrodes (FIG. 2, top panel) and presents a side-view potential energy diagram (FIG. 2, bottom panel) of how the tunneling current may be thought of during such an event.

In one embodiment, the tunneling chip, a 0.5 mm silicon substrate, is coated by vapor deposition with 50 nm of silicon nitride. In addition to being readily wetted by aqueous solutions, silicon nitride is mechanically robust. It has high dielectric strength (10⁷ volts/cm) and dc resistivity (10¹⁴ ohm-cm) [10]. The metal electrodes are placed by photo-lithography or

electron-beam lithography as are well-known in the art [11]. Electrical connection points for the tunneling electrodes are exposed at the top face of the tunneling chip. The thickness of the insulating layer between the metal electrodes is a critical consideration because of the exponential decay of the wave function. It is initially set at 2 nm, but successive chip fabrication runs may readily be varied to explore the method's sensitivity to this parameter. In certain embodiments, the insulator is chosen from among: silicon dioxide, silicon nitride or hafnium-based oxides. The last is recommended based on its higher electrical resistivity. Correct device fabrication is verified by scanning electron microscopy.

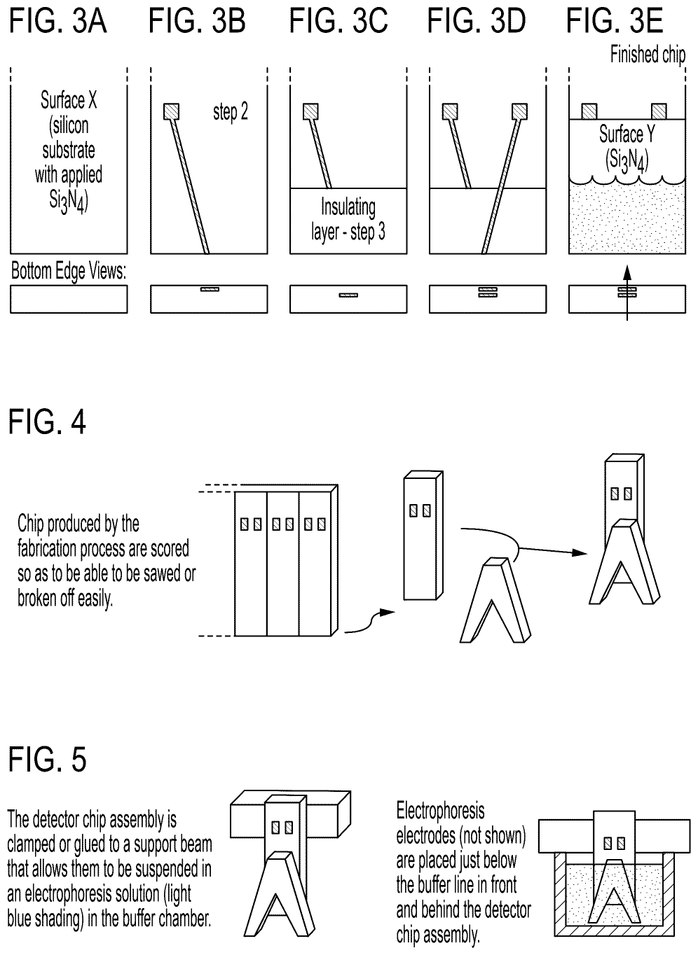

The fabrication steps are shown in FIGS. 3A-3E. Roughness in the tunneling current electrodes as a result of chip dicing in the fabrication process can be eliminated by electrochemical polishing of excess gold extrusion from the chip edge [12], or in the alternative, if a depression has been created in the gold electrode sensing surface, deposition of additional conducting material in a final electroplating or “smoothing” fabrication step [13]. The presence of any defects, and their subsequent correction, can be determined and verified by scanning electron microscopy.

In the process of fabrication, two rows of 6 chips each are laid out on a 100 mm wafer. The silicon wafer above and below those rows is discarded leaving a 6×8 cm rectangle (two rows of 6×4 cm) with the remaining arc of the wafer at each end for handling.

The finished chip is referred to here as the “tunneling chip”. It can be clamped or glued behind a second plastic or Teflon™ module (the “notch chip”) having any of various comb or notch channel geometries. This is shown in FIG. 4. This modular design allows a rapid “mix-and-match” in establishing the best working combination for a particular application.

It will be readily understood by those skilled in the art that there are many methods for sample introduction into the electrophoresis system described in this invention. One example is that a “blue” 1000 μl disposable pipette tip is sealed at its tip with 10-20 μl of low percentage

ultra-pure, RNAse-free agarose gel [14]. Atop this gel, the sample, in glycerol solution, is introduced and then carefully overlaid with electrophoresis buffer. The negative electrode is inserted in this buffer, and the tip/plug/sample/buffer/electrode is then placed or clamped into position on one side of the detector assembly in the chamber. Electrophoresis begins with the nucleic acid extruded from the tip, moving down across the tunneling chip (with its measurement electrodes) and then up toward the positive electrode(s) above and on the other side of the notch. This method is presented by way of example and not limitation to the full scope of this invention. Other sample introduction possibilities exist, and special attention is accorded to those that are simple and readily allow for automation.

The present invention is inspired by earlier work with doughnut hole circular, solid-state nanopores [15]. In contrast, the importance of the new, open structure invented here is two-fold. First, it prevents the problem of spontaneous nanopore closure [16] or clogging [17]. Second, it overcomes the very short nucleic acid transit times observed through the circular, solid-state nanopores.

Under typical experimental conditions, DNA moves through circular nanopores at rates of about 10 base pairs per microsecond [18]. It follows then that, with good signal-to-noise ratios, the detection system bandwidth must be in excess of 10 megahertz if there is to be any chance at all of identifying nucleotide bases. Unfortunately, commercially available systems such as the Axopatch 200B—traditionally used for electrophysiology measurements, but frequently used now in nanopore experiments—have an operating bandwidth of only about 50 kilohertz. Improvements to this performance have been made in a new generation of current amplifiers specifically engineered for nanopore studies [19]. These amplifiers employ custom CMOS circuitry that provides sampling rates of 40 million samples per second with a dynamic range of +/−100 nA (10 MHz Nanopore reader). With lower noise, the Elements 100 kHz Nanopore reader, used in these studies, provides a sampling rate of over 100 thousand measurements per second with RMS noise of about 20 pA observed when used according to the manufacturer's instructions and shielded in a Faraday cage.

Still, in spite of advances in measurement technology, solid-state nanopores remain a poor choice for nucleic acid sequencing because of the rapid movement of the nucleic acid polymer through the nanopore. The solution to this problem, described in the invention here, is to do away with the closed nanopore. By increasing the effective area experiencing the electric field and through which the nucleic acid moves under the electrophoresis, transit time for the polymer is increased.

The theory of electrophoresis allows an estimate of how much slowing in transit time between a conventional closed circular nanopore and an open nano-notch might be expected. Following from Van Holde's example [20], if an element of volume in the electrophoretic cell has a cross-section A and thickness dx, and if the potential difference across that slab is dV, then by Ohm's law,

$$dV = - idR \qquad ( 1 )$$

where −i is the current (flowing toward the lower potential) and dR is the resistance of the slab of solution being considered. dR can be rewritten as dx/KA, where K is the specific conductance of the solution in the volume slab. In addition, −dV/dx is the potential gradient, E, and therefore,

$$E = i / KA \qquad ( 2 )$$

If the nucleic acid's speed of transit through the nanopore, v, is proportional to the potential gradient, E, then for the same solution and electrophoresis conditions operating on the same nucleic acid and moving only through different geometries, one can divide Equation (2) for v₁ corresponding to A₁ by the same equation written for v₂ and A₂, giving upon simplification,

$${v}_{1} / {v}_{2} \approx {A}_{2} / {A}_{1} \qquad ( 3 )$$

Equation (3) is written as an approximation because the placement of the electrodes in the experiments here do not produce a uniform potential gradient across the electrophoretic aperture, as is typical in a nanopore experiment. Rather, in the present invention, the electrodes are positioned so as to pull the nucleic acids through the comb- or notch-like structure in tight contact with the sensing electrodes in the tunneling chip at the root. Still, if the diameter of an experimental nanopore is 5 nm, then an effective electrophoretic cross-sectional area of 1×10⁻¹² m²—a square micron—experienced by a nucleic acid at the fork root is expected from Equation (3) to reduce transit velocity by at least 4 orders of magnitude.

Besides the dimensions of the electrophoresis aperture, solution and temperature conditions have also been shown to be able to decrease nucleic acid transit speeds in nanopores, but only though by about a factor of ten [21].

The decreased speed of transit of the nucleic acid across the tunneling current electrodes is sufficient to allow the electronics to “see” the transit of the nucleic acid and measure the duration of its presence. In many cases, for particular samples, it is possible during the transit of the nucleic acid, to reverse the polarity of electrophoresis (under computer control) and cause the nucleic acid to shuttle back and forth across the tunneling electrodes.

The present invention employs a roughly cubic electrophoresis chamber, approximately 2 cm on a side and open at top. The chamber rests in a temperature-controlled water bath. The electrophoresis electrodes are placed on opposite sides of the detector chip assembly (tunneling chip+notch chip), within the single pool of buffered electrophoresis liquid, but above the notch or fork channel that is expected to be traversed by the nucleic acid molecule.

FIG. 5 diagrams the overall arrangement. When an assay is completed, the detector chip assembly is lifted and placed into an adjacent cleaning/sterilizing solution (not shown). In actual operation, this sort of arrangement allows a cap to then be placed on the used (possibly contaminated) electrophoresis solution chamber. Applying a slight vacuum, as with a syringe, seals the cap, and the whole assembly can be removed from the water bath for biohazard disposal. This operation after a detection run, and then the replacing of a fresh, pre-filled electrophoresis chamber, and reintroduction of the cleaned detector chip assembly is best accomplished under robotic control for automation.

Calibration experiments with this system can employ commercially available, single-stranded RNAs, such as the purine homopolymer poly(A) or the pyrimidine homopolymer poly(C) [22]. Using these simplifies the interpretation of the signals observed. In addition, if reversing the polarity of electrophoresis proves impractical, using homopolymers wherein all of the bases to be measured are the same makes it possible to overlay datasets and thus simulate the effect of an increased electrical signal sampling rate. This provides data for estimating the sampling rate necessary in experiments for base identification at various nucleic acid transit speeds. Known amounts of these homopolymers, or other nucleic acids of known sequence, can also be introduced into the tip containing the sample so as to provide a calibration standard or as a positive control.

Duplex DNAs in aqueous solution have as many as 17 water molecules associated with each residue [23]. Single-stranded RNAs will have fewer, but still perhaps enough to mask base- or modified base-distinctive chemical features from the tunneling sensors under some experimental conditions. Mixed aqueous/organic electrophoresis solutions and two-phase solvent electrophoresis systems, such as buffered H₂O:CCl₄, in which the root of the fork is partially submerged in an immiscible, dehydrating solvent after the initiation of electrophoretic transit of the polynucleotide, are easily accessible. The use of such solvent systems lie within the scope of the present invention.

Heroic efforts have been made to improve the bandwidth of the electronics available to researchers for nanopore studies [24]. Integrating CMOS amplifiers onto the nanopore chip has increased bandwidths to approximately 500 kilohertz and additional advances in this area may be possible [25]. Critical to this improvement is the minimization of parasitic impedance by placing the amplification electronics as close as possible to the sensing surfaces of the tunneling electrodes [7].

Besides improvements to the electronics, the effective sampling rate can also be increased by incorporating multiple sets of tunneling electrode sensors into a single electrophoretic channel. In this way, the nucleic acid transiting the channel can effectively be looked at multiple times in a single passage. The idealized device in FIG. 1 shows two sets of tunneling sensors. There is no reason, however, why tens or perhaps even hundreds of these pairs cannot be incorporated into a single channel, and multiple channels be used in a single electrophoresis chamber, resulting in massive parallelism and redundancy in data acquisition. Embodiments such as these lie within the scope of this invention.

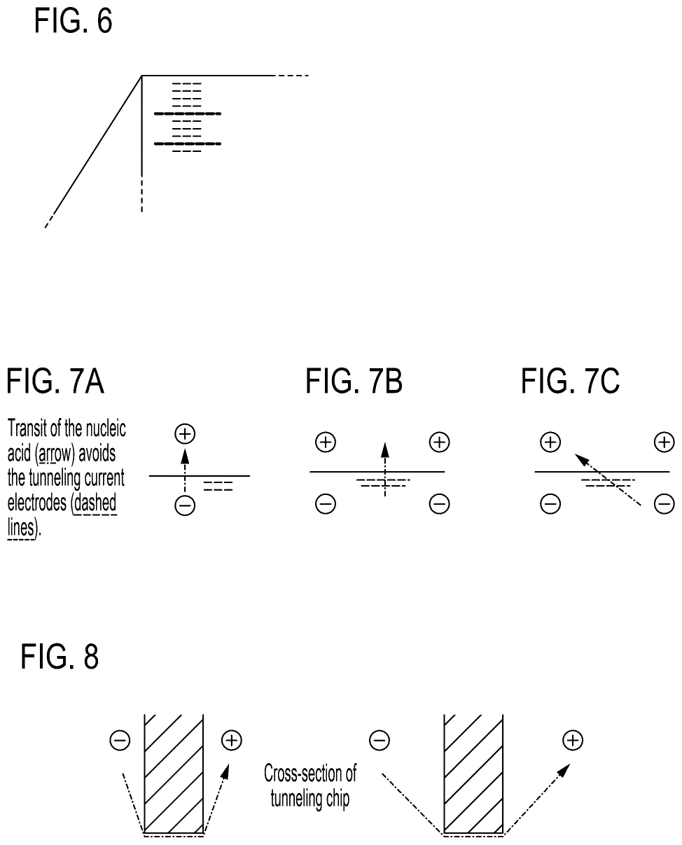

Two additional considerations further recommend the use of multiple sets of tunneling electrodes:
1.) The forces of Brownian motion on a nucleic acid molecule in solution may cause it to back up across the detecting electrodes. Error frequency produced by this molecular “double vision” will vary with electrophoresis conditions, but it cannot be a priori eliminated. Multiple sets of detecting electrodes in the same channel can detect and correct this phenomenon. These sets are diagrammatically represented in FIG. 6 and, as stated above, such configurations with their detection software lie within the scope of this invention.
2.) DNA has a well-known double helical structure, but single-stranded RNAs also exhibit helical twisting along their axes[26]. This twisting can cause interpretable signals to fade-in and fade-out with the transit of the molecule in a corkscrew-like fashion across a given electrode pair. Again, multiple pairs of detecting electrodes can overcome this problem.

In addition to multiple pairs of tunneling current detection electrodes, the present invention provides for additional electrodes, separately powered by and working with the electrophoresis voltage, to be incorporated into the structure as diagrammed in FIG. 6.

Analogous to the rightmost panel of FIG. 1, and not to scale, the pairs of tunneling current sensing electrodes are each represented by a single light blue line; and the “hold and release” electrodes are shown as the longer, thicker green lines. Both of these are drawn at the top face (also, the “working face”) of the channel root, reflecting their introduction by vapor deposition/nanolithography on one surface of the original silicon substrate. Both faces can be used, however, and multiple chips stacked, limited by the size, mass, and heat generation of the associated amplification and A/D conversion electronics.

During a nucleic acid's transit, these electrodes can be energized and biased, under computer control, so as to briefly hold the molecule in place against the driving electrophoretic force. If a number of them are present, a process can be set up in which their polarity is alternated so as to “hold and release” the nucleic acid in a stepwise fashion and “inch worm” the molecule through the channel without other electrophoretic voltage.

An operational concern with this invention's design is that because the length of the tunneling electrodes is only a small percentage of the total notch or comb width, many molecules under electrophoresis may be able to shoot through the opening without passing over those electrodes. This situation is diagrammed in FIG. 7A. The solution to this problem, provided by this invention, is to make the shortest distance between the electrophoresis electrodes lie directly over the tunneling current detection electrodes. In addition, however, there is an operational solution to this problem. Multiple positive and negative electrophoresis electrodes may be placed into the conductive solution on each side of the comb or notch channel. If the electrophoresis voltage is partitioned between them, it is possible to create a net electric field vector that causes the molecule to pass directly across the tunneling electrodes. This arrangement is shown diagrammatically in FIG. 7B.

In a first approximation, as shown in FIG. 7B, the nucleic acid moves perpendicularly across the electrodes. Initial experiments will typically set the insulator thickness at 2 nm (Tᵢₙ). From the geometry of the system, however, it is clear that the thickness of the insulator experienced by the molecule (Tₑₓ) depends on the angle, α, of its crossing the insulator gap, such that Tₑₓ=Tᵢₙ/cos(α). As an example then, if the net electrophoresis field vector is adjusted in such a way as to cause the nucleic acid to move at 45° across tunneling electrodes separated by a 2 nm insulator, as diagrammed in FIG. 7C, the effective tunneling distance will be ˜2.8 nm.

Finally, as diagrammed in FIG. 8, the electrophoresis electrodes may be placed closer or farther away from the faces of the detector chip assembly. This results in a variable “angle of pull” of the polymer molecule (shown in red) through the channel. Besides affecting the molecule's degree of contact with the tunneling electrode pairs (and perhaps limiting damage), the electrodes' repositioning will also affect the electric field intensity and influence molecular transit times. The electrodes' positioning, of course, need not be symmetrical.

Table 1 summarizes the performance outcomes from one embodiment of the invention described here.

TABLE 1
Outcomes.

| | | Time necessary to obtain sequence with 5X redundancy in the base sequence determination | | | | | |
|---|---|---|---|---|---|---|---|
| System | | Covid | | Bacterial ID | | Human genome | |
| Performance | | (~30 kb*) | | (1 Mb^{†}) | | (6.4 × 10⁹ bp^{‡}) | |
| 1 | kb/sec | 2.5 | min | 1.4 | hours | 370 | days |
| 4 | kb/sec | 38 | sec | 21 | min | 93 | days |
| 1 | Mb/sec | 0.15 | sec | 5 | sec | 8.9 | hours |
| 10 | Mb/sec | 15 | msec | 0.5 | sec | 53 | min |

*kb = kilobases = 1000 bases.
^{†}Mb = million bases.
^{‡}bp = base pairs.

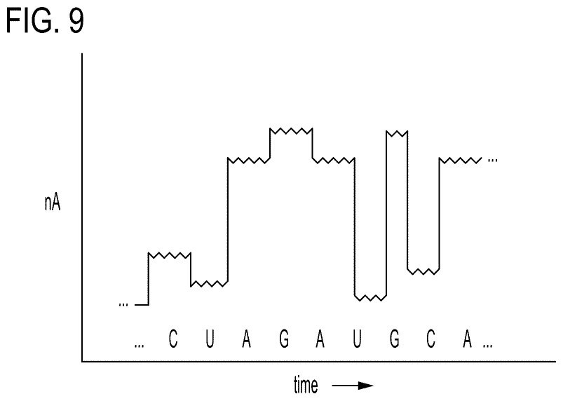

FIG. 9 shows an idealized data output and base assignment interpretation of the signals obtained from the operation of the invention.

There is increasing interest in the biology of circular RNAs (CircRNA) in human cells [28] and the human microbiome [29], and correspondingly in methods for sequencing these molecules [30]. For analyses by the present invention, CircRNAs can be linearized by hybridizing to a single-stranded oligonucleotide DNA sequence having the restriction enzyme recognition site (and appropriate flanking sequences, usually redundant) of an enzyme known to nick the RNA strand of an RNA: DNA heteroduplex [31].

### REFERENCES CITED AND INCORPORATED WITH THEIR REFERENCES INTO THE SPECIFICATION

[1] Deamer, D., Akeson, M., and Branton, D. Three decades of nanopore sequencing. Nat. Biotechnol. 34, 518-524 (2016) and Xue, L., Yamazaki, H., Ren, R., Wanunu, M., Ivanov, A. P., and Edel, J. B. Solid-state nanopore sensors. Nat. Reviews Materials 5, 931-951 (2020).
[2] Kasianowicz J., Brandin, E., Branton, D., and Deamer, D. Characterization of individual polynucleotide molecules using a membrane channel. Proc. Natl. Acad. Sci. USA 93, 13770-13773 (1996) and Church, G., Deamer, D. W., Branton, D., Baldarelli, R., and Kasianowicz, J. Characterization of individual polymer molecules based on monomer-interface interactions. U.S. Pat. No. 5,795,782 (1998).
[3] Bhattacharya, S., Derrington, I. M., Pavlenok, M., Niederweis, M., Gundlach, J. H., and Aksimentiev, A. Molecular dynamics study of MspA arginine mutants predicts slow DNA translocations and ion current blockades indicative of DNA sequence. ACS Nano 6, 6960-6968 (2012).
[4] Jain, M., Fiddes, I. T., Migal, K. H., Olsen, H. E., Benedict Paten, B., and Akeson, M. Improved data analysis for the MinION nanopore sequencer. Nat. Methods 12, 351-356 (2015).
[5] Burdick, J. T., Comai, A., Bruzel, A., Sun, G., Dedon, P. C., and Cheung, V. G. Nanopore-based direct sequencing of RNA transcripts with 10 different modified nucleotides reveals gaps in existing technology. G3 13, 1-12 (2023). World-wide-web at https://doi.org/10.1093/g3journal/jkad200.
[6] Deamer, D., Akeson M., and Branton, D. in their response to Kasianowicz and Bezrukov. Nat. Biotechnol. 34, 482, col. 3 (2016).
[7] Rosenstein, J., Ray, V., Drndic, M., and Shepard, K. L. Solid-state nanopores integrated with low-noise preamplifiers for high-bandwidth DNA analysis. IEEE Xplore Digital Library, pp. 59-62 (2011). doi: 10.1109/LISSA.2011.5754155
[8] Simmons, J. G. Generalized formula for the electric tunnel effect between similar electrodes separated by a thin insulating film. J. Appl. Phys. 34, 1793-1803 (1963); and Electric tunnel effect between dissimilar electrodes separated by a thin insulating film. ibid. 34, 2581-2590.
[9] Banerjee, S. and Zhang, P. Review of recent studies on nanoscale electrical junctions and contacts: quantum tunneling, current crowding, and interface engineering. J. Vac. Sci. Technol. A 40, 030802 (2022). doi: 10.1116/6.0001724.
[10] Sze, S. M. Physics of Semiconductor Devices second edition, John Wiley & Sons, New York (1981).
[11] Levinson, Harry J., and Society of Photo-optical Instrumentation Engineers, publisher. Principles of Lithography (4^{th} edition). SPIE (2019) and Brewer, et al. Electron-Beam Technology in Microelectronic Fabrication. Academic Press, New York, NY (1980).
[12] Zaki, S., Zhang, N., and Gilchrist, M. D. Electropolishing and shaping of micro-scale metallic features. Micromachines 13, 468-503 (2022). doi.org/10.3390/mil3030468.
[13] Huang, J-F. and Fan, M-C. Micro-morphological reconstruction of nanoporous gold electrode. J. Mater. Chem. 20, 1431-1434 (2010).
[14] Thermo Fisher Scientific, ultra-pure low melting point agarose on the world-wide-web at thermofisher.com/order/catalog/product/16520050.
[15] Lin, B., Hui, J., and Mao, H. Nanopore technology and its applications in gene sequencing. Biosensors 11, 214 (2021) and Magierowski, S., Huang, Y., Wang, C., and Ghafar-Zadeh, E. Nanopore-CMOS interfaces for DNA sequencing. Biosensors 6, 42 (2016). c.) Venkatesan, B. M. and Bashir, R. Nanopore sensors for nucleic acid analysis. Nat. Nanotechnol. 6, 615-624 (2011). and d.) Branton, D., et al. The potential and challenges of nanopore sequencing. Nat. Biotechnol. 26, 1146-1153 (2008).
[16] The atoms surrounding small diameter nanopores have significant surface energy. This allows them to move around. It is observed that they appear to be able to move into nanopore openings and close them.
[17] In biological isolates where proteins have not been removed, anionic proteins such as serum albumin, R_{G} (in solution)≈3 nm (see ref. 20, p. 196), may electrophoretically migrate to clog nanopores.
[18] Oliver, J. S. and Dimitrov, V. Close encounters: integrating nanopores and CMOS amplifiers for single-molecule detection. Nat. Methods 9, 453-454 (2012).
[19] Elements SRL on the world-wide-web at elements-ic.com.
[20] Van Holde, Kensal Edward. Physical Biochemistry. Prentice-Hall, Englewood Cliffs, New Jersey (1971). See p. 127.
[21] Fologea, D., Uplinger, J., Thomas, B., McNabb, D. S., and Li, J. Slowing DNA translocation in a solid state nanopore. Nano Lett. 5, 1734-1737 (2005).
[22] Millipore Sigma catalog:
   poly(A) P9403 on world-wide-web at www.sigmaaldrich.com/US/en/product/sigma/p9403.
   poly(C) P4903 on world-wide-web at www.sigmaaldrich.com/US/en/product/sigma/p9403.
   poly(G) P4404 on world-wide-web at www.sigmaaldrich.com/US/en/product/sigma/p4403.
[23] The number of waters bound/associated depends on how they are measured. See: Saenger, W. Water and nucleic acids. In: Principles of micleic acid structure. Springer Advanced Texts in Chemistry, Springer, New York, NY (1984). In regard to nucleic acid bases, see: Furmanchuk, A., Isayev, O., Shishkin, O., Gorb, L., and Leszczynski, J. Hydration of nucleic acid bases: a Car-Parrinello molecular dynamics approach. Phys. Chem. Chem. Phys. 12, 3363-3375 (2010).
[24] Rosenstein, J. K. (2013). Noise optimization for high-bandwidth ion channel recordings. [PhD thesis, Columbia University]. Retrieved from academiccommons.columbia.edu/doi/10.7916/D8PKOPC9.
[25] Rosenstein, J., Wanunu, M., Merchant, C., Drndic, M., and Shepard, K. L. Integrated nanopore sensing platform with sub-microsecond temporal resolution. Nat. Methods 9, 487-492 (2012). Summarized in ref. 18 and further reviewed in ref. 15b. See also the patent: U.S. Pat. No. 9,217,727 B2 Dec. 22, 2015.
[26] Eichhorn, C. D. and Al-Hashimi, H. M. Structural dynamics of a single-stranded RNA-helix junction using NMR. RNA 20, 782-791 (2014). doi.org/10.1261/rna.043711.113.
[27] The length of the example nucleic acid strand (in red in FIG. 8) is greatly exaggerated and is pictured here as a non-limiting example. The smallest human chromosome has ˜47 million base pairs and a calculated length of about 1.6 cm. See human chromosome 21 description at en. wikipedia.org/wiki/Human_genome.
[28] Cao, Q. M., Boonchuen, P., Chen, T-C., Leid, S., Somboonwiwat, K. and Sarnowa, P. Virus-derived circular RNAs populate hepatitis C virus-infected cells. Proc. Natl. Acad. Sci. USA 121 (7) e2313002121. doi.org/10.1073/pnas.2313002121.
[29] Zheludev, I. N., Edgar, R. C., Lopez-Galiano, M. J., de la Peña, M., Babaian, A., Bhatt, A. S. and Fire, A. Z. Viroid-like colonists of human microbiomes. bioRxiv doi: doi.org/10.1101/2024.01.20.576352 (2024).
[30] Adkar-Purushothama, C. R. and Perreault, J-P. Impact of Nucleic Acid Sequencing on Viroid Biology. Int. J. Mol. Sci. 21, 5532-5556 (2020). doi: 10.3390/ijms21155532
[31] Murray, I. S., Stickel, S. K., and Roberts, R. J. Sequence-specific cleavage of RNA by Type II restriction enzymes. Nucleic Acids Res. 38, 8257-8268 (2010). And see the supplemental data thereto. doi: 10.1093/nar/gkq702

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of characterizing the composition of a charged polymer comprising
introducing the polymer into an electrically conductive medium,
applying a voltage to said conductive medium that causes said polymer to move across closely spaced measurement electrodes, wherein none of said measurement electrodes comprises a closed circular structure, so that a tunneling current between said closely spaced measurement electrodes is produced,
measuring time-dependent changes in said tunneling current, and
correlating said changes in said tunneling current to the composition of said polymer.

2. The method of claim 1, wherein the polymer is a polynucleotide.

3. The method of claim 2, wherein the polynucleotide is substantially single-stranded.

4. The method of claim 1, wherein the polymer is a polypeptide.

5. The method of claim 1, wherein the closely spaced measurement electrodes have been processed by the electrodeposition of a conducting material on their surfaces.

6. The method of claim 1, wherein the first derivative of the time-dependent change in the tunneling current is calculated to determine the composition of the polymer.
